(19) United States
(12) Patent Application Publication
(10) Pub. No.: US 2023/0080692 A1
(43) Pub. Date: Mar. 16, 2023

(54) COMPOSITION FOR USE IN TREATING OSTEOARTHRITIS

(71) Applicant: Meluha Therapeutics Sdn. Bhd, Putrajaya (MY)

(72) Inventors: Premasangery Kathivaloo, Putrajaya (MY); Nur Aina Sahira Zainal Azian, Putrajaya (MY); Ramesh Saravanamuthu, Putrajaya (MY)

(21) Appl. No.: 17/877,797

(22) Filed: Jul. 29, 2022

(30) Foreign Application Priority Data
Sep. 7, 2021 (MY) PI2021005162

(51) Int. Cl. A61K 35/28; A61K 31/728; A61P 19/02

(52) U.S. Cl. CPC A61K 35/28; A61K 31/728; A61P 19/02

(57) ABSTRACT

The present invention relates to a composition for use in treating degenerative joints disorders in a patient in need thereof. The degenerative joints disorder is osteoarthritis (OA). The composition comprises mesenchymal stem cell (MSCs) and Hyaluronic acid (HA).

### CROSS-REFERENCE TO RELATED APPLICATIONS

This application claims priority to Malaysian Patent Application No. PI2021005162 filed Sep. 7, 2021, which is incorporated by reference in its entirety for all purposes.

### FIELD OF THE INVENTION

The present invention relates to a composition for use in treating degenerative joints disorders in a patient in need thereof, in particular the degenerative joints disorder is osteoarthritis.

### BACKGROUND OF THE INVENTION

Osteoarthritis is abbreviated as OA or referred to as degenerative arthritis or degenerative joint disease (DJD). OA is a form of arthritis that features the breakdown and eventual loss of the cartilage of one or more joints. Cartilage is a protein substance that serves as a “cushion” between the bones of the joints. As the cartilage breaks down, the ends of the bones thicken and may begin to rub together and eventually causing harsh pain.

The changes that occur in the joint are brought about by changes in the quality and amount of the hyaluronic acid (HA) in the joint. Hyaluronic acid is the main component of joint cartilage and synovial fluid. HA shows high viscosity and elastic property, which maintains healthy joints. Sufferers of degenerative arthritis do not produce sufficient hyaluronic acid and accelerated degeneration results in the destruction of hyaluronic acid, decreasing its concentration and molecular weight. The synovial joint, particularly the large weight-bearing joint contains hyaluronic acid which has lubricating and shock-absorbing properties, allowing the joints to move normally and painlessly. In patients with degenerative joints disorders, such as osteoarthritis, the viscoelasticity of the synovial fluid is markedly reduced with a decrease in the lubricating and shock-absorbing functions. This results in a mechanical loading of the joint and the degeneration of the joint cartilage (restricted and painful joint movement). The administration of highly purified hyaluronic acid can improve the viscoelastic properties of the synovial joint. The lubricating and shock-absorbing properties of HA reduces pain and improves mobility of joints.

As mesenchymal stem cells (MSCs) have capacity to differentiate into bone and cartilaginous tissue, orthopedists have chosen regenerative medicine using MSCs as an option for the treatment of chronic conditions, such as joint cartilage lesions and arthritis. Typically, supportive biomaterials are used to mimic the extracellular matrix architecture of the specific tissue which assist the cell proliferation and differentiation. In most cases, HA is used as a biomaterial because HA is the main component of the extracellular matrix and significantly contributes to cell proliferation differentiation due to its lubricating and shock-absorbing properties. Hence, the current treatment for OA patients is intra-articular injection therapy with MSC and HA wherein the MSC and HA given as separate injection to the OA patients.

The above-described method has its own disadvantage as the HA needs to be administered separately which is undesirable to the OA patients as it causes additional pain and discomfort to the OA patients. Thus, there is a need to develop a suitable composition for treating osteoarthritis that could overcome the above raised problems as well as be desirable to OA patients.

### SUMMARY OF THE INVENTION

The present invention discloses a composition for use in treating OA wherein the composition comprises MSCs and Hyaluronic acid (HA). The MSCs is used in an amount ranging between 15.0×10⁶ to 25.0×10⁶ of cells, preferably between 18.0×10⁶ to 22.0×10⁶ of cells. The HA is used in an amount ranging between 1.0% to 2.0%, preferably between 1.3% to 1.5% by total weight of the of a media for culturing the composition for use in treating OA. The media for culturing the composition for use in treating OA is a complete culture medium (CCM) comprising Knockout-Dulbecco's Modified Eagle Medium (KO-DMEM), serum, L- glutamine, aminoglycoside antibiotic and growth factor.

Additional aspects, features and advantages of the invention will become apparent to those skilled in the art upon consideration of the following detailed description of preferred embodiments of the invention in conjunction with the drawing listed below.

### BRIEF DESCRIPTION OF THE ACCOMPANYING DRAWINGS

The present invention will be fully understood from the detailed description given herein below and the accompanying drawings which is given by way of illustration only, and thus is not limitative of the present invention, wherein:

In the appended drawings:

FIG. 1 illustrates control replicates comprises WJSC and DPBS and experimental replicates comprises WJSC, DPBS and HA.

FIG. 2 illustrates control replicates comprises WJSC and HA and experimental replicates comprises WJSC, CCM and HA.

FIG. 3 illustrates control replicates cultured on T75 flasks comprises WJSC and CCM and experimental replicates cultured on T75 flasks comprises WJSC, CCM and HA for a clearer understanding.

FIG. 4 illustrates control replicates comprises WJSC, CCM, HA and experimental replicates comprises WJSC, CCM and HA for (i) chondrogenic differentiation and (ii) osteogenic differentiation.

FIG. 5 illustrates WJSC growth cultured with HA observed under microscope using 4× magnification.

FIG. 6 illustrates flow cytometry analysis of WJSC (Control) labelled with antibodies CD73, CD90, CD105 and Negative markers.

FIG. 7 illustrates flow cytometry analysis of WJSC+HA labelled with antibodies CD73, CD90, CD105 and Negative markers.

FIG. 8 illustrates chondrogenic and osteogenic differentiation of WJSC cultured with HA using 4× magnification.

### DETAILED DESCRIPTION OF THE INVENTION

Detailed description of preferred embodiments of the present invention is disclosed herein. It should be understood, however, that the embodiments are merely exemplary of the present invention, which may be embodied in various forms. Therefore, the details disclosed herein are not to be interpreted as limiting, but merely as the basis for the claims and for teaching one skilled in the art of the invention. The numerical data or ranges used in the specification are not to be construed as limiting.

Mesenchymal stem cells” or “MSCs” refers to multipotent stem cells capable of differentiating into osteoblasts, chondrocytes, myocytes, adipocytes, neuronal cells, pancreatic islet cells, and the like. In more detail, mesenchymal stem cells are multipotent stem cells located in the Wharton's Jelly Stem Cells (WJSC), bone marrow, peripheral blood, adipose tissue but not limited thereto.

The present invention relates to a composition for use in treating degenerative joints disorders in a patient in need thereof, in particular, the degenerative joints disorder is osteoarthritis (OA).

First aspect of the present invention discusses on a composition for use in treating OA wherein the composition comprises MSCs and Hyaluronic acid (HA).

The MSCs is used in an amount ranging between 15.0×10⁶ to 25.0×10⁶ of cells, preferably between 18.0×10⁶ to 22.0×10⁶ of cells. The MSCs is selected from the group consisting of WJSC, bone marrow derived stem cells, peripheral blood stem cells, adipose tissue derived stem cells and any combinations thereof, preferably WJSC.

The HA is used in an amount ranging between 1.0% to 2.0%, preferably between 1.3% to 1.5% by total weight of a media for culturing the composition for use in treating OA.

Second aspect of the present invention discusses on a media for culturing the composition for use in treating OA wherein the media is a complete culture medium (CCM). The CCM comprises Knockout-Dulbecco's Modified Eagle Medium (KO-DMEM), serum, L- glutamine, aminoglycoside antibiotic. Gentamicin and growth factor.

The KO-DMEM is used in an amount ranging between 80.0% to 91.0%, preferably between 87.0% to 90.0%, most preferably 88.82% by total weight of the CCM.

The serum is used in an amount ranging between 5.0% to 12.0%, preferably between 8.0% to 11.0%, most preferably 10.0% by total weight of the CCM. The serum used is Fetal Bovine Serum.

The L- glutamine is used in an amount ranging between 0.5% to 1.2%, preferably between 0.7% to 1.1%, most preferably 1.0% by total weight of the CCM. The L-glutamine used is Glutamax.

The antibiotic is used in an amount ranging between 0.05% to 0.12%, preferably between 0.08% to 0.11%, most preferably 0.10% by total weight of the CCM. The antibiotic used is Gentamicin.

The growth factor is used in an amount ranging between 0.05% to 0.10%, preferably between 0.07% to 0.09%, most preferably 0.08% by total weight of the CCM. The growth factor used is Basic Fibroblast Growth Factor (BFGF).

Table 1 shows chemical components of the CCM of the present invention and compositions thereof.

TABLE 1
Chemical components of the CCM of the present
invention and compositions thereof

| Chemical | Working | Preferred | Typical |
|---|---|---|---|
| components | range (%) | range (%) | value (%) |
| KO-DMEM | 80.0 to 91.0 | 87.0 to 90.0 | 88.82 |
| Serum | 5.0 to 12.0 | 8.0 to 11.0 | 10.0 |
| L- glutamine | 0.5 to 1.2 | 0.7 to 1.1 | 1.0 |
| Aminoglycoside antibiotic | 0.05 to 0.12 | 0.08 to 0.11 | 0.10 |
| Growth Factor | 0.05 to 0.10 | 0.07 to 0.09 | 0.08 |

The third aspect of the present invention discloses a method of isolating and cultivating the WJSC, preferably the WJSC at subculture 3 (SC3). The harvested WJSC of subculture 2 (SC2) are cultured with Complete Culture Medium (CCM) in a T175 cm² culture flask. Once the WJSC reach 80%±5% confluency, SC3 WJSC are harvested and resuspended in a 1 ml CCM to perform cell count using a Countess Automated Cell Counter.

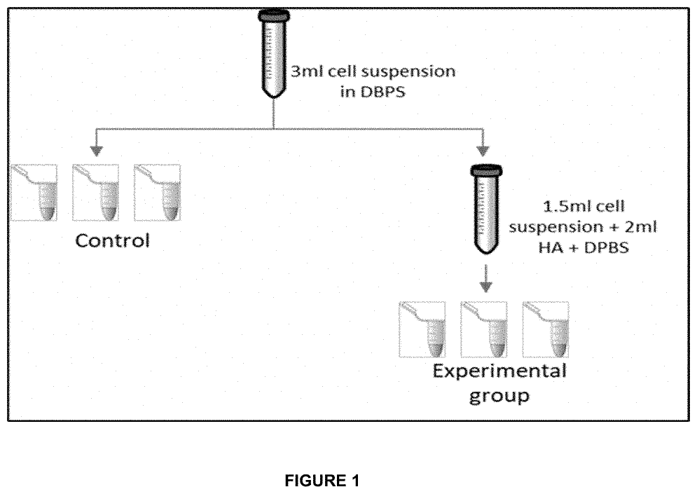

The fourth aspect of the present invention discloses a method of resuspending WJSC with HA. Approximately 16,500,000 WJSC in SC3 were resuspended in 3 ml Dulbecco's phosphate-buffered saline (DPBS) and divided into control and experimental group. Each group were divided into three replicates. Then, 0.5 mL of the solution was aliquot into 3×1.5 ml microcentrifuge tubes. The tubes were labelled as control. Then, 2 mL of HA was diluted with DPBS and resuspended with the remaining cell. The cell suspension was aliquot equally (around 1 ml) into 3×1.5 ml microcentrifuge tubes. The tubes were labelled as Experimental 1 (E1) and Experimental 2. FIG. 1 illustrates control replicates comprises WJSC and DPBS and experimental replicates comprises WJSC, DPBS and HA for a clearer understanding. Table 2 shows compositions of the experimental groups and replicates thereof (as mentioned above). Cell viability analysis was performed at an interval of 4, 24, and 48 h of incubation at room temperature using Countess Automated Cell Counter.

TABLE 2
Composition of the experimental groups and replicates thereof

| Group | Composition | Replicate |
|---|---|---|
| Control | WJSC + DPBS | 3 |
| Experimental | WJSC + DPBS + HA | 3 |

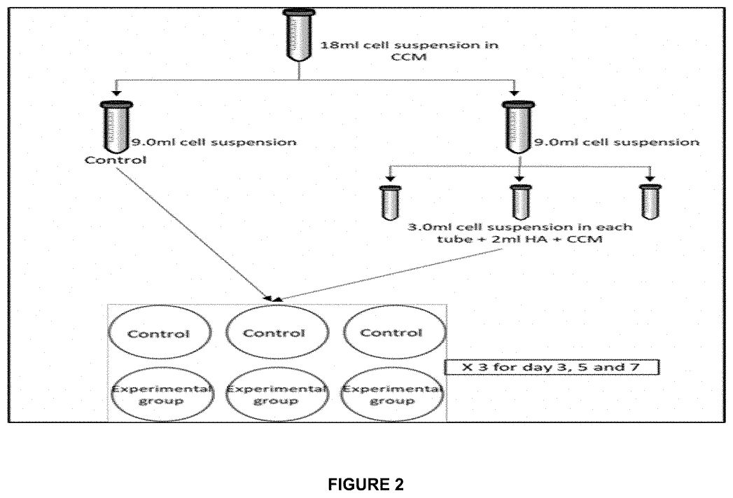

The fifth aspect of the present invention discusses on WJSC cultured with HA. Approximately 900,000 SC2 WJSC were resuspended with 18 ml CCM and divided into control and experimental group. Each group were divided into three replicates. Then, 9 ml of WJSC suspension (approximately 450,000) was aliquot for control and the remaining suspension divided into three units of 50 mL tube (3 mL/tube) for treatment purpose. Next, 1 ml of WJSC suspension was aspirated from the control tube and seeded in a 6 well plate. For the experimental group, 2 mL of HA was added into each of the tubes containing 3 mL of WJSC suspension. The WJSC suspension with HA in each tube was mixed and a few mL of CCM added if the suspension is still viscous. All the WJSC suspension was aspirated and seeded equally into three well of 6 well plate. All three unit of six well-plate were incubated at the temperature of 37° C. with 5% CO₂. FIG. 2 illustrates control replicates comprises WJSC and HA and experimental replicates comprises WJSC, CCM and HA for a clearer understanding. Table 3 shows compositions of the experimental groups and replicates thereof (as mentioned above). Cell growth and proliferation was observed, harvested and cell count was conducted on days 3, 5 and 7.

TABLE 3
Composition of the experimental groups and replicates
and number of well plates thereof

| | | | Number of |
|---|---|---|---|
| Group | Composition | Replicate | well plate |
| Control | WJSC + CCM | 3 | 3 |
| Experimental | WJSC + CCM + HA | 3 | 3 |

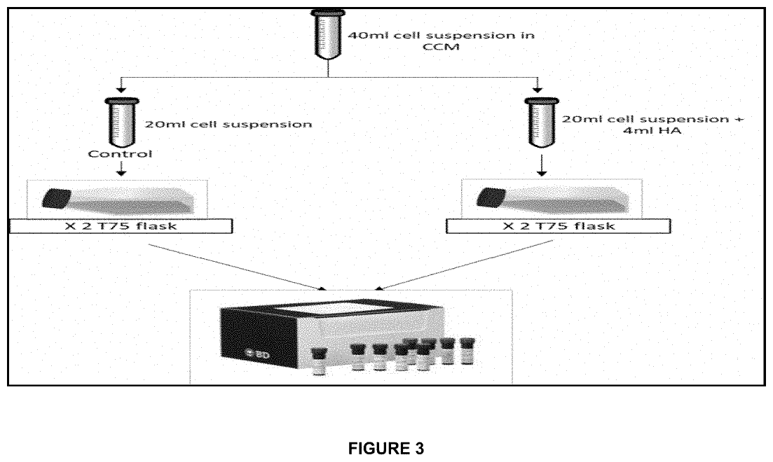

The sixth aspect of the present invention discusses on method of immunophenotyping of WJSC cultured with HA. Approximately 1,500,000 SC2 WJSC were resuspended with 40 ml CCM and divided into control and experimental group (each 20 ml WJSC suspension). For control, the WJSC were cultured in 2× T75 flasks with 10 ml WJSC suspension respectively. Whereas, for the experimental group the 20 ml WJSC suspension was resuspended with 4 ml HA and cultured in 2× T75 flasks with 10 ml WJSC suspension respectively. After 7 days, the WJSC were harvested and the surface markers of WJSC were analyzed using Human MSC Analysis Kit antibodies (Becton-Dickinson, USA). The WJSC were stained with hMSC Positive Cocktail (CD73, CD90 and CD105) and hMSC negative Cocktail (CD45, CD38, CD19, CD11 b and HLA-DR). Stained WJSC were kept in dark and incubated for 30 minutes on ice before analyzed for marker expression using BD Flow Cytometry System FACS Calibur. FIG. 3 illustrates control replicates cultured on T75 flasks comprises WJSC and CCM and experimental replicates cultured on T75 flasks comprises WJSC, CCM and HA for a clearer understanding.

The seventh aspect of the present invention discusses on differentiation of WJSC cultured with HA. Approximately 600,000 SC2 WJSC resuspended with 12 ml CCM and divided equally into two units of 50 mL tube (6 mL/tube). Then, 4 mL of HA was added into each of the tubes containing 6 mL of WJSC suspension. The WJSC suspension with HA was mixed and a few mL of CCM added if the suspension is still viscous to obtain WJSC solution. Thereafter, the cell solution subjected for (i) chondrogenic differentiation and (ii) osteogenic differentiation. The details are as below:

I. Chondrogenic Differentiation

1 ml of WJSC solution from one of the tubes was cultured in the center of the well to generate micro-mass culture. Additional 3 mL of CCM was added to culture the WJSC until it reached 80±5% confluence. Prior to chondrogenesis induction, Complete Chondrogenesis Differentiation Medium (CDM) and staining solution of Safranin O was prepared. After the WJSC reach 80% confluence, the CCM media was discarded and washed with 5 mL of DPBS twice. Then, 2 mL of pre-warmed CDM was added and continued incubation for 21 days. The CDM was changed every 4 days. After 21 days, CDM was removed and rinsed with 5 mL DPBS twice. The WJSC were fixed with 4% PFA solution for 30 min and incubated at 18±2° C. for 10 minutes. Then, rinsed the wells with DPBS and stained the WJSC with 2 mL Safranin O solution for 30 minutes at room temperature. Before visualizing the WJSC under a light microscope, rinsed the well three times with DEPC water and captured the image for analysis. Present of red staining indicates the synthesis of proteoglycans by chondrocytes.

II. Osteogenic Differentiation

1 ml of WJSC solution from one of the tubes was cultured in the center of the well to generate micro-mass culture. Additional 3 mL of CCM was added to culture the WJSC until it reached 80±5% confluence. Prior to osteogenesis induction, Complete Osteogenesis Differentiation Medium (ODM) and staining solution of 0.2% Alizarin Red was prepared. After the WJSC reach 80% confluence, the CCM media was discarded and washed with 5 mL of DPBS twice. Then, 2 mL of pre-warmed ODM was added and continued incubation for 21 days. The ODM was changed every 4 days. After 21 days, CDM was removed and rinsed with 5 mL DPBS twice. The WJSC were fixed with 4% PFA solution for 30 min and incubated at 18±2° C. for 10 minutes. Then, rinsed the wells with DPBS and stained the WJSC with 2 mL Alizarin Red solution for 30 minutes at room temperature. Before visualizing the WJSC under a light microscope, rinsed the well three times with DEPC water and captured the image for analysis. Presence of black calcium deposit staining indicates the synthesis of osteogenesis.

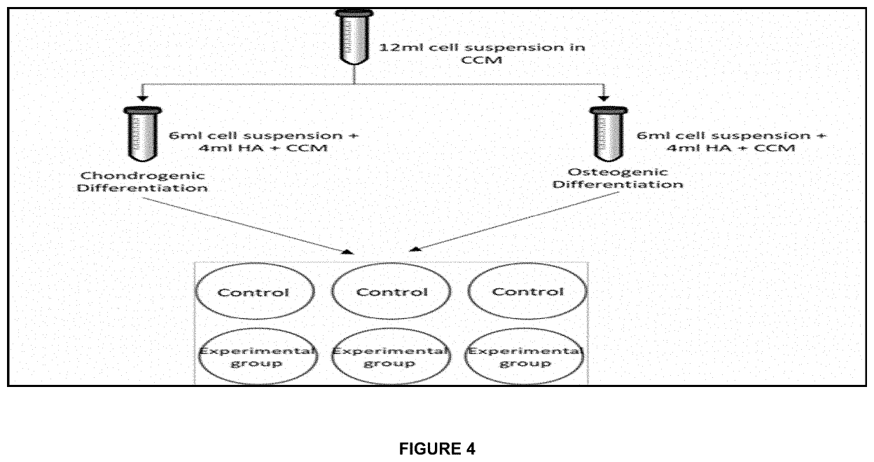

FIG. 4 illustrates control replicates comprises WJSC, CCM, HA and experimental replicates comprises WJSC, CCM and HA for (i) chondrogenic differentiation and (ii) osteogenic differentiation for a clearer understanding. Table 4 shows compositions of the control replicates comprises WJSC, CCM, HA and experimental replicates comprises WJSC, CCM and HA for (i) chondrogenic differentiation and (ii) osteogenic differentiation (as mentioned above).

TABLE 4
Composition of the control replicates comprises
WJSC, CCM, HA and experimental replicates comprises
WJSC, CCM and HA for (i) chondrogenic differentiation
and (ii) osteogenic differentiation

| Group | Composition | Replicate |
|---|---|---|
| Control | WJSC + CCM + HA (No induction) | 3 |
| Chondrogenic | WJSC + CCM + HA | 3 |
| Differentiation | | |
| Control | WJSC + CCM + HA (No induction) | 3 |
| Osteogenic | WJSC + CCM + HA | 3 |
| Differentiation | | |

The WJSC cultured with HA is subjected to analysis such as:
(i) viability rate as discussed in the fourth aspect of the present invention
(ii) growth and proliferation rate as discussed in the fifth aspect of the present invention
(iii) immunophenotyping analysis as discussed in the sixth aspect of the present invention and
(iv) differentiation analysis, as discussed in the fifth aspect of the present invention details of which are discussed below.

Viability Rate

The initial cell count in each tube is 2,750000 cells/ml. Table 5 shows viability of WJSC exposed to HA for 48 hours.

TABLE 5
Viability of WJSC exposed to HA for 48 hours

| | Group | | | |
|---|---|---|---|---|
| | Control | | Experimental | |
| Hours | Mean ± SD | Viability (%) | Mean ± SD | Viability (%) |
| 4 hours | 2.33 ± 0.25 | 63 | 2.32 ± 0.13 | 82 |
| 24 hours | 2.20 ± 0.29 | 60 | 2.30 ± 0.08 | 81 |
| 48 hours | 2.13 ± 0.38 | 60 | 2.27 ± 0.12 | 78 |

All the values are mean±SD; n=3 in each group. The mean of cell count are millions of cells/ml.

Based on the results showed in Table 5, it has proven that WJSC were viable in HA after 48 hours of incubation at room temperature. Further, it was observed that cell viability in the experimental group was better than the control.

Growth and Proliferation Rate

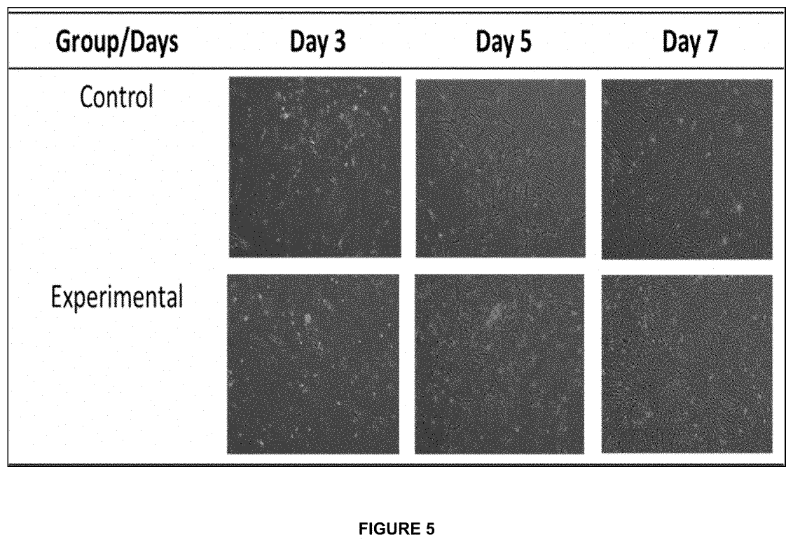

The number of WJSC seeded per well is 50,000 cells. FIG. 5 illustrates WJSC growth cultured with HA observed under microscope using 4× magnification. The WJSC displayed the typical fibroblast-like spindle shape morphology when viewed under an inverted microscope. The homogenous monolayer of WJSC attached in plastic flask able to proliferate and achieve 80-90% confluency in 7 days. There are no changes observed between the morphology of WJSC and WJSC cultured with HA. The WJSC were able to maintain the fibroblast spindle shape morphology in both the groups.

Table 6 shows viability of WJSC exposed to HA for 7 days.

TABLE 6
Viability of WJSC cultured with HA for 7 days

| | Group | | | |
|---|---|---|---|---|
| | Control | | Experimental | |
| Day | Mean ± SD | Viability (%) | Mean ± SD | Viability (%) |
| 3 days | 0.024 ± 0.0092 | 54 | 0.012 ± 0.0029 | 55 |
| 5 days | 0.040 ± 0.0050 | 63 | 0.022 ± 0.0030 | 72 |
| 7 days | 0.082 ± 0.0020 | 87 | 0.062 ± 0.0035 | 86 |

All the values are mean ± SD; n = 3 in each group. The mean of cell count are millions of cells/ml.

In term of cell proliferation, it was observed that the cell count for WJSC cultured with HA has comparable viability with control (WJSC only).

Immunophenotyping Analysis

Human MSC Analysis Kit antibodies which consist of positive marker (CD73, CD90 and CD105) and negative marker (CD45, CD38, CD19, CD11 b and HLA-DR) were used to characterize the immunophenotypes of isolated WJSC and WJSC cultured with HA. Based on the analyzed marker expression, both WJSC and WJSC cultured with HA shows high expression of stromal specific markers for MSCs and low expression of endothelial and hematopoietic markers, which indicate pure characteristic of the MSCs population. The immunophenotypes were analysed using flow cytometry and the results were presented in Table 7.

TABLE 7
Immunophenotyping results for
WJSC and WJSC cultured with HA

| Markers/Antibodies | Control | Experimental |
|---|---|---|
| Negative marker | 0.0% | 0.0% |
| CD73 | 99.7% | 99.8% |
| CD90 | 99.7% | 99.7% |
| CD105 | 59.7% | 63.7% |

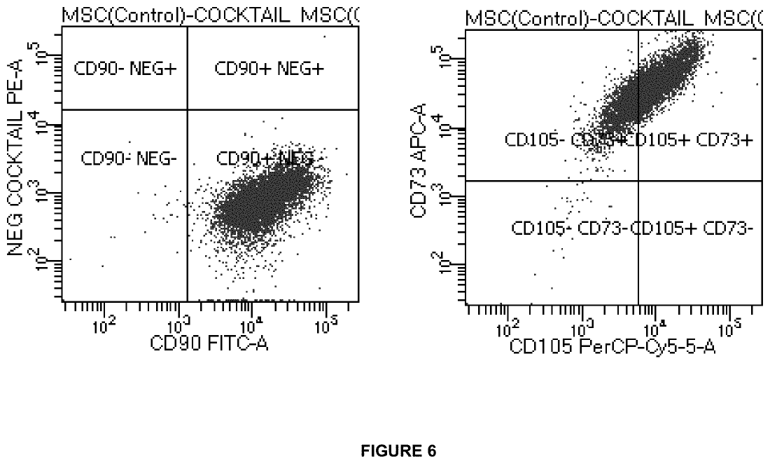

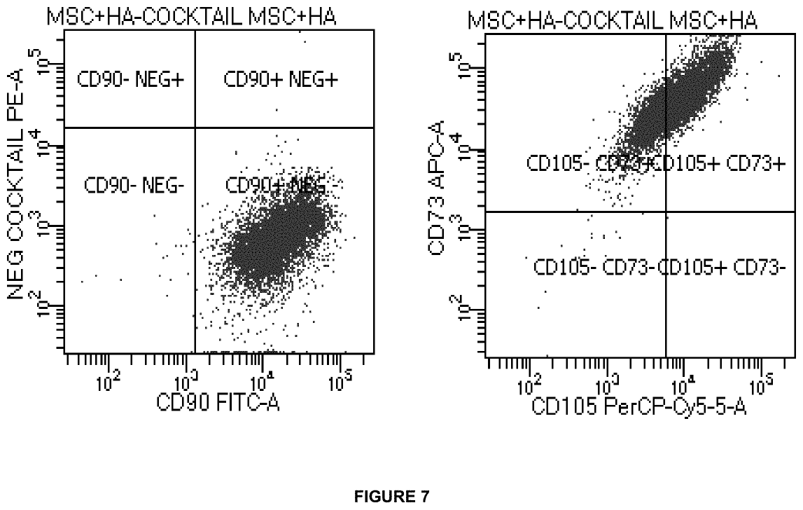

FIG. 6 illustrates flow cytometry analysis of WJSC (Control) labelled with antibodies CD73, CD90, CD105 and Negative markers. Meanwhile, FIG. 7 illustrates flow cytometry analysis of WJSC+HA labelled with antibodies CD73, CD90, CD105 and Negative markers.

The flow cytometry demonstrated that WJSC cultured with HA did not affect the expression of the MSC-associated markers compared to controls. Only slight variations in the positive expression of CD105 were observed following the treatment of MSC with HA.

Differentiation Analysis

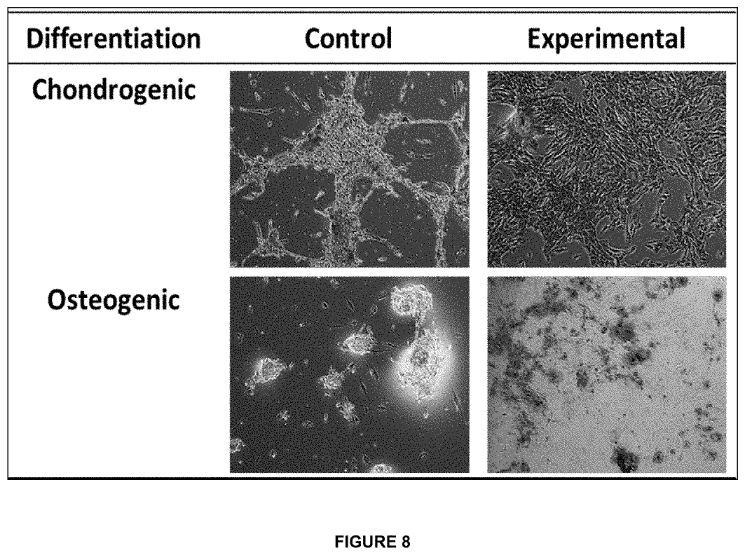

FIG. 8 illustrates chondrogenic and osteogenic differentiation of WJSC cultured with HA (4× magnification). Chondrogenic induction differentiation was confirmed by Safranin O staining, where the red staining indicates the synthesis of proteoglycans by chondrocytes. Osteogenic induction differentiation was confirmed by the presence of black calcium deposits through Alizarin Red staining which indicates synthesis of osteogenesis. The results obtained shows that WJSC cultured with HA maintain its MSC characteristic and potential for multipotent in vitro differentiation.

Based on the outcome, it is evident that the WJSC cultured with HA enables proliferation, maintains its morphology and MSC characteristics and is able to differentiate into osteogenesis and chondrogenesis. Therefore, it can be concluded that HA is safe to be cultured with WJSC for therapeutic application. The composition for use in treating osteoarthritis of the present invention can be administered in a single injection to a patient in need thereof. Thus, the composition of the present invention can be used for the manufacture of a medicament for treating OA, wherein the medicament is administered in a therapeutically effective amount in one single injection, in a patient in need thereof.

In view of the above, the composition for use in treating osteoarthritis of the present invention overcomes the problems of MSC and HA wherein each of the compound (MSC and HA) of the composition had to be administered as a separate injection to the OA patients. The results obtained in the present invention show that HA is safe to be cultured with WJSC for therapeutic application. Also, it is evident that the composition for use in treating osteoarthritis of the present invention can be administered in a single injection to a patient in need thereof.

The terminology used herein is for the purpose of describing example embodiments only and is not intended to be limiting. As used herein, the singular forms “a”, “an” and “the” may be intended to include the plural forms as well, unless the context clearly indicates otherwise. The terms “comprises”, “comprising”, “including” and “having” are inclusive and therefore specify the presence of stated features, integers, steps, operations, elements and/or components, but do not preclude the presence or addition of one or more other features, integers, steps, operations, elements, components and/or groups therefrom.

1. A composition for use in treating joint disorder comprising mesenchymal stem cell, MSC and Hyaluronic acid, HA, wherein the HA is in an amount ranging between 1.0% to 2.0% by total weight of a media for culturing the composition for use in treating OA wherein the joint disorder is osteoarthritis.

2. The composition for use in treating OA as claimed in claim 1, wherein the media for culturing the composition is a complete culture medium, CCM comprising Knockout-Dulbecco's Modified Eagle Medium, KO-DMEM, serum, L- glutamine, aminoglycoside antibiotic and growth factor.

3. The composition for use in treating OA as claimed in claim 2, wherein the KO-DMEM is used in an amount ranging between 80.0% to 91.0% by total weight of the CCM.

4. The composition for use in treating OA as claimed in claim 2, wherein the serum is used in an amount ranging between 5.0% to 12.0% by total weight of the CCM.

5. The composition for use in treating OA as claimed in claim 2, wherein the L-glutamine is used in an amount ranging between 0.5% to 1.2% by total weight of the CCM.

6. The composition for use in treating OA as claimed in claim 2, wherein the antibiotic is used in an amount ranging between 0.05% to 0.12% by total weight of the CCM.

7. The composition for use in treating OA as claimed in claim 2, wherein the growth factor is used in an amount ranging between 0.05% to 0.10% by total weight of the CCM.

8. Use of the composition as claimed in claim 1 for the manufacture of a medicament for treating OA, wherein the medicament is administrable in a therapeutically effective amount, in a patient in need thereof.

9. Medicament for treating joint disorders wherein the medicament includes the composition as claimed in claim 1.
